(19) United States
(12) Patent Application Publication
(10) Pub. No.: US 2010/0143325 A1
(43) Pub. Date: Jun. 10, 2010

(54) Composition And Methods Involving Thrombolytic Agents

(75) Inventor: Victor Gurewich, Cambridge, MA (US)

Correspondence Address: MEDLEN & CARROLL, LLP, 101 HOWARD STREET, SUITE 350, SAN FRANCISCO, CA, 94105, US

(73) Assignee: Vascular Laboratory, Inc.

(21) Appl. No.: 12/330,557

(22) Filed: Dec. 9, 2008

(51) Int. Cl. A61K 38/48; A61P 9/00

(52) U.S. Cl. 424/94.64

(57) ABSTRACT

Treatment or prevention methods are described wherein t-PA and C1-inhibtor are used together in order to minimize the hemorrhagic complications of tPA Preferably, C1-inhibitor is infused prior to treatment with t-PA, thereby allowing for a safer thrombolysis without the excessive and dangerous bleeding associated with the use of t-PA alone particularly in the treatment of ischemic stroke.

### FIELD OF THE INVENTION

The invention generally relates to thrombolysis, and more specifically, to the treatment and/or prevention of cardiovascular disease, including but not limited to the treatment and/or prevention of stroke comprising t-PA and C1-inhibitor. In a preferred embodiment, methods and compositions are provided to reduce bleeding (intracranial) from t-PA during the treatment of ischemic stroke.

### BACKGROUND

Cardiovascular disease is the number one cause of death in the United States. Medical Sciences Bulletin, No. 238; p. 1 (1997). While cardiovascular disease presents many different clinical manifestations, myocardial infarction and stroke are two of the major ones.

Stroke is the second most common cause of death worldwide (Murray and Lopez, 1997); ischemic stroke due to a thrombotic or embolic occlusion is responsible for approximately 80% of all strokes. Thrombolysis, referring to dissolution of the occluding thrombus, represents the only available pharmacological modality for reversing or limiting the brain damage in ischemic stroke. Thrombolysis with recombinant tissue plasminogen activator (rt-PA), is the only thrombolytic treatment approved when given within three hours of symptoms onset. However, due to bleeding side effects, and in particular a ten-fold increase in symptomatic intracranial haemorrhage, the clinical use of tPA has been limited to about 1-2% of eligible patients (The NINDS rt-PA Stroke Study Group, 1995). Moreover, since these hemorrhagic side effects are iatrogenic, dramatic, and often fatal, they represent a major impediment for the clinician to use tPA.

### SUMMARY OF THE INVENTION

The invention generally relates to thrombolysis, and more specifically, to the treatment of cardiovascular disease, including but not limited to the treatment of stroke and heart attack with formulations comprising t-PA (unmutated and mutated forms of t-PA).

In a preferred embodiment, methods and compositions are provided to reduce this bleeding (intracranial) side effect of t-PA during the treatment of ischemic stroke. In one embodiment, the present invention contemplates treatment with t-PA and C1-inhibtor. These two components can be administered together (e.g. a single solution comprising both compounds) or separately (e.g. two solutions). However, in a preferred embodiment, exogenous C1-inhibitor is infused prior to treatment with t-PA. For example, in one embodiment, the present invention contemplates a method of treating: a) providing: i) a subject having symptoms of ischemia; ii) a first solution comprising C1-inhibitor, and iii) a second solution comprising tissue plasminogen activator; b) infusing said subject with at least a portion of said first solution; and c) infusing said subject, after step b), with at least a portion of said second solution, under conditions wherein said symptoms are reduced (or reversed). In one embodiment, the subject is a human and has symptoms of (or has been diagnosed as having) a stroke (or heart attack). In one embodiment, said infusing is intravenous infusion. In another embodiment, said infusing is intra-arterial (e.g. for intra-arterial thrombolysis). Both t-PA and C1-inhibitor may be recombinantly produced.

Whereas the reduction in symptoms and brain damage by tPA will not be reduced, it is believed that the associated side-effects will be reduced. As noted previously, the major side-effect with t-PA is bleeding (specifically intracranial haemorrhaging which can cause death). As shown herein, the use of C1-inhibitor prior to treatment with t-PA reduces bleeding significantly (and consequently reduces the risk of mortality). This reduction in bleeding may permit administration to subjects who otherwise would be unsuitable for t-PA treatment alone. Thus, in one embodiment, the present invention contemplates a method of reducing the risk of intracranial bleeding, comprising: a) providing: a subject having symptoms of ischemic stroke; a first solution comprising tissue plasminogen activator, and a second solution comprising C1-inhibitor; b) infusing said subject with at least a portion of said second solution prior to said first solution, in order to reduce the risk of intracranial bleeding caused by tissue plasminogen activator (without significantly impairing its therapeutic effect); c) infusing said subject, after step b), with at least a portion of said first solution. In one embodiment, there is no significant impairing of its therapeutic effect where thrombolysis is achieved.

As noted above, the present invention, in one embodiment, contemplates preventing or reducing ischemia with tPA more safely. In one embodiment, C1-inhibitor and t-PA are employed in subjects at risk for ischemia. Thus, in one embodiment, the present invention contemplates a safer method of preventing the extension of ischemia after thromboembolic occlusion and preventing the ischemic zone becoming irreversibly infarcted comprising: a) providing: i) a subject with ischemia due to a thromboembolic occlusion ii) a first solution comprising C1-inhibitor, and iii) a second solution comprising tissue plasminogen activator; b) infusing said subject with at least a portion of said second solution; and c) infusing said subject, after step b), with at least a portion of said first solution, under conditions wherein the risk of ischemic damage is reduced. In an alternative embodiment, the t-PA and C1-inhibitor is given simultaneously. Thus, in one embodiment, the present invention contemplates a formulation, comprising a mixture of tissue plasminogen activator and C1-inhibitor, said mixture formulated for infusion (e.g. intravenous infusion).

In one embodiment, the present invention contemplates a method of reducing the risk of brain infarction, comprising: a) providing: i) a subject at risk for infarction from an occlusive thrombus; ii) a first solution comprising C1-inhibitor, and iii) a second solution comprising tissue plasminogen activator, b) infusing said subject with at least a portion of said first solution; c) infusing said subject, after step b), with at least a portion of said second solution, under conditions wherein the risk of infarction from an ischemic stroke is reduced. In one embodiment, said subject is a human with ischemic stroke at risk of infarction due to an occlusive thrombus. In an alternative embodiment, the tPA and C1-inhibitor is given simultaneously.

In one embodiment, the present invention contemplates a method of reversing ischemia, comprising: a) providing: i) a subject at risk for ischemia from a non-occlusive thrombus; ii) a first solution comprising C1-inhibitor, and iii) a second solution comprising tissue plasminogen activator; b) infusing said subject with at least a portion of said first solution; c) infusing said subject, after step b), with at least a portion of said second solution, under conditions wherein the risk of brain damage from ischemia is reduced. In one embodiment, said subject is a human at risk for an ischemic stroke due to a non-occlusive thrombus. In another embodiment, said subject is a human at risk for a heart attack due to a non-occlusive thrombus.

It is not intended that the present invention be limited by the particular nature of a preparation or solution. Among the physiologically acceptable compositions for use in the methods is physiological saline or phosphate buffered saline, in which the agents are dissolved or suspended, such that the resulting composition is suitable for infusion. Formulations may contain such normally employed additives as binders, fillers, carriers, preservatives, stabilizing agents, emulsifiers, buffers and excipients as, for example, pharmaceutical grades of mannitol, lactose, starch, magnesium stearate, sodium saccharin, cellulose, magnesium carbonate, and the like. These compositions typically contain 1%-95% of active ingredient, preferably 2%-70%.

### DEFINITIONS

As used herein, the term “subject” refers to both humans and animals.

As noted above, the present invention contemplates treating and preventing brain damage from ischemia. As used herein, “ischemia” is a restriction in blood supply so that less blood is delivered to an organ. Ischemia is a potentially reversible state and can be detected by MRI. If it is not reversed, it will go on to infarction, which is the death of tissue. Ischemia in the heart produces the symptom of chest pain or angina. Ischemia in the brain reduces brain function, producing a variety of neurological symptoms, including confusion, loss of speech, loss of movement (e.g. in the limbs), impaired vision, and the like. Prolonged ischemia can cause permanent brain damage and irreversible symptoms. Severe and prolonged ischemia in the brain can result in death. An ischemic “stroke” is ischemia in the brain caused by a blot clot.

As used herein, the phrase “wherein one or more symptoms are reduced” refers to a qualitative or quantitative reduction in one or more detectable symptoms, including but not limited to a detectable impact on the rate of recovery from an ischemic incident. For example, treatment according to the present invention may reduce limb paralysis. In a preferred embodiment, treatment according to the present invention increases blood flow by dissolving the blot clot causing ischemia. Symptoms are “reduced” when blood flow is restored or improved. It is not intended that the present invention be limited only to cases where the symptoms are eliminated. The present invention specifically contemplates treatment such that symptoms are reduced (and the condition of the subject is thereby “improved”), albeit not completely eliminated. For example, there may be, depending on the case, some residual brain or heart damage, depending upon the speed with which the underlying ischemia is reversed.

Determination whether an adult human is suffering from cardiovascular disease including stroke is readily made by a person skilled in the art using a number of readily available diagnostic procedures. These include history, physical examination, CT scan and MRI as well as certain blood tests.

As used herein, “C1-inhibitor” and “C1-INH” refer to C1 esterase inhibitor, that contains the C1-INH serpin domain. This is a serum alpha-2 globulin and a member of the serpin family of protease inhibitors that is synthesized by the liver, but may be produced by recombinant technology in transgenic animals or from prokaryote or eukaryote cells or the C1INH serpin domain may be included in a chimeric molecule. Its physiologic function is inhibition of the catalytic subunits of the first component of the classic complement pathway (C1r and C1s). In certain embodiments, the dose of exogenous (infused) C1-inhibitor for humans is between 50 U/kg and 200 U/kg, and more typically 100 U/kg, which may be delivered as a bolus. Native human C1INH purified from plasma is available and marketed by Lev Pharmaceutical and has been approved for the treatment of hereditary angioedema in the US and Europe. Recombinantly produced C1-inhibitor from transgenic rabbits is produced by Pharming (Leiden, NL) and has been submitted for approval in Europe.

In one embodiment, the present invention also contemplates using a “C1INH-type protein” which is a protein comprising the serpin domain (also referred to as a “serpin reactive center loop,” or a “center reactive loop”) comprising amino acid residues 98 through the C-terminus of C1INH, but lacking other parts of the native C1-inhibitor molecule.

As used herein, “t-PA” is tissue plasminogen activator. Genentech produces t-PA under the name Activase® (Alteplase) which is tissue plasminogen activator produced by recombinant DNA technology. The present invention contemplates unmutated and mutated forms. Some mutated forms have been made to improve the half-life See e.g. EP0293934, incorporated herein by reference.

Unmutated t-PA is available commercially (recombinantly produced) from Genentech. A variety of doses of t-PA can be used, however, for stroke administered intravenously at a dose of 0.9 mg/kg or less. Importantly, in some embodiments, a dose 1-1.2 mg/kg is permitted, provided the subject has been previously infused with C1-inhibitor.

Mutated t-PA is also available commercially and contemplated for use together with C1-inhibitor, in the various manners described herein. Genetech provides Tenecteplase (TNK) which is t-PA that has been altered with amino acid substitutions at three different positions of the molecule, in the T, N and K domains. The substitutions prolong the half-life. Doses of 0.25 mg/hr to 0.5 mg/hr are reported to be effective.

Mutated t-PA is also available from Centacor under the name Retevase (Reteplase) (Ret). In the case of this variant, the finger, epidermal growth factor, and kringle 1 regions have been deleted. It is reported to have lower fibrin binding than unmutated t-PA.

### DESCRIPTION OF THE INVENTION

The invention generally relates to thrombolysis, and more specifically, to the treatment of cardiovascular disease, including but not limited to the treatment of stroke with formulations comprising thrombolytic agents. In a preferred embodiment, methods and compositions are provided to reduce bleeding (intracranial) from t-PA during the treatment of ischemic stroke. In one embodiment, the present invention contemplates treatment with t-PA and C1-inhibtor. These two components can be administered together (e.g. a single solution comprising both compounds) or separately (e.g. two solutions). However, in a preferred embodiment, exogenous C1-inhibitor is infused prior to treatment with t-PA.

Stroke is the third most common cause of death in the United States, and the leading cause of disability. More than 700,000 Americans suffer strokes each year, and about 170,000 of them die. tPA is the only treatment available and FDA-approved to reduce the incidence and extent of disability from ischemic stroke. And yet, a recent report indicates that forty (40) percent of emergency physicians say they're unlikely to give stroke patients tPA even in an ideal setting, mostly because of the fear of causing brain bleeding. However, if the risk of bleeding associated with tPA could be reduced, more physicians said they would use it. The findings are from a published survey of 1,105 emergency physicians conducted by University of Michigan Stroke Program researchers and published early online in the Annals of Emergency Medicine. The report indicates there is still controversy over the use of tPA, or tissue plasminogen activator. Most importantly, the report clearly show that there are patients who need t-PA that are not getting it.

The current reluctance to use t-PA for stroke is understandable. A brain hemorrhage caused by treatment is an iatrogenic event which has a more devastating impact on all concerned than a natural event. Currently, in-hospital mortality is higher for stroke patients treated with tPA compared with patients not receiving tPA. When experience with thrombolytic therapy is limited, the risk of adverse events and in-hospital mortality is even higher. Heuschmann et al., Stroke 34:1106 (2003).

The present invention provides, in one embodiment, a way to address the problem by providing a safer protocol for t-PA administration and one that may permit extending the permissible treatment period beyond the current three-hour limit. In one embodiment, the present invention contemplates treatment or prevention with t-PA together with C1-inhibitor. These two components can be administered together (e.g. a single solution comprising both compounds) or separately (e.g. two solutions). However, in a preferred embodiment, exogenous C1-inhibitor is infused prior to stroke treatment with t-PA.

In this manner C1-inhibitor helps to control the potentially deadly side-effects of t-PA alone. This positive effect of C1-inhibitor with tPA in animals with ischemic stroke is surprising and could not have been envisioned from the only very modest inhibition of tPA (single-chain or two-chain) by C1-inhibitor reported by Huisman et al, Thromb Haemost. 73, 466-71 (1995). Since C1-inhibitor does not interfere with the therapeutic effect of tPA, the dose of C1-inhibitor can be varied can permit some variation in the dose of t-PA in order to enhance its efficacy. Currently, the accepted protocol for t-PA alone to treat stroke involves giving approximately 0.9 mg/kg, 10% as a bolus, intravenously. Also, currently one is not to exceed the 90 mg maximum dose due to hemorrhagic side effects even though it is known that higher doses provided superior efficacy (Braunwald E, Knatterrud G L, Passamani E R, Robertson T L Announcement of protocol change in Thrombolysis in Myocardial Infarction Trial. J Am Coll Cardiol 1987;9:467). However, in one embodiment, the use of C1-inhibitor together with t-PA (preferably prior to t-PA) permits higher dosing and higher maximum dosages for greater efficacy.

### Experimental

The following examples serve to illustrate certain preferred embodiments and aspects of the present invention and are not to be construed as limiting the scope thereof.

### Materials

M5 is a site-directed mutant (Lys 300 to His) of pro-urokinase (proUK) constructed and expressed in Escherichia coli as previously described (Liu et al., 2002) and obtained by Primm (Milan, Italy). Single-chain tissue plasminogen activator (t-PA), pharmaceutical grade, was purchased from Genentech (San Francisco, Calif., USA). Recombinant proUK expressed in E. coli was obtained from Landing Science and Technology Company, Nanjing, China. Aprotinin was obtained as Trasylol form Miles, Inc. (Kankakee, Ill., USA). Berinert P® (Behring GmbH, Germany) was used as the source for C1-inhibitor; alteplase (Actilyse® 50 mg, Boehringer Ingelheim GmbH, Germany) was adopted as source of normal, non mutant t-PA was a generous gift of dr. Bergui.

### Surgical Procedures

All experimental procedures on live rats were performed according to the European Communities Council Directive (86/609/EEC) and with the guidelines for care and use of laboratory animals as published by Italian Ministry of Health (DDL 116/92). Animals had free access to food and water. All efforts were made in order to minimize the number of animals used and their suffering. Rectal temperature was maintained at 36.5-37.5° C. throughout the procedure with a heating pad. Male adult Sprague-Dawley rats (Harlan, Italy), weighing 250 to 350 g, were used to assess different thrombolytic drug activity after ischemic damage. Permanent ischemia was induced using the procedure previously described by Renoulleau et al. (1998). Briefly, rats were anaesthetised with 2% isoflurane vaporised in a 70/30 mixture of N₂O/O₂ using a face mask. Under the operating microscope (Carl Zeiss Inc., Thornwood, N.Y., USA) a midline incision of the head was performed, the temporal muscle dissected and the temporal bone exposed; a burr hole was drilled very close to the zygomatic arch and the left middle cerebral artery (MCA) was identified. MCA main branch was then electrocoagulated close to its origin at the junction with olfactory branch. After this procedure, a median incision was made in the neck to expose the left common carotid artery (CCA) and a clip was placed to occlude it, in order to reduce infarction size variability due to anastomoses in the MCA territory (Renolleau et al., 1998). After 90 minutes, clip was removed and reperfusion allowed.

### Drug Administration Protocols

Rats were randomly allocated into five groups, each one receiving different thrombolytic treatments: group 1 (n=7) received t-PA 10 mg/kg, the standard dose in rat stroke models in the literature. Ten percent was given as a bolus and the remainder as an intravenous infusion through the femoral vein over 30 minutes using an infusion pump (KD Scientific, Holliston, Mass., USA). Group 2 (n=8) was treated with the same protocol as above with the addition of a intravenous bolus of C1-inhibitor (100 U/kg) prior to the t-PA infusion; group 3 (n=5) was infused with M5 (15 mg/kg over 30 minutes, preceded by 10% as bolus) together with C1-inhibitor as above; vehicle-only infusion was given to group 4 (n=5), while group 5 (n=5) received C1-inhibitor only. Drug administration was performed in all animals four hours after ischemia onset, since this is the therapeutic window for thrombolysis eligibility in humans (Schellinger et al., 2001). The dose of M5 was based on that which gave equivalent clot lysis in rat plasma in vitro. C1-inhibitor was given routinely after it was determined that rat C1-inhibitor did not interact with tcM5, in contrast with human C1-inhibitor.

In this study, rt-PA was infused four hours after ischemia onset, which is within the therapeutic window for thrombolysis in humans (PROACT study; The NINDS rt-PA Stroke Study Group, 1995) This delay probably helps explain the higher mortality than was reported at this dose by most studies in the literature. (Jiang et al., 2004; Rasmussen et al., 2003). In rats, t-PA must be given at a dose ten-times higher than in humans to obtain comparable effects (Korninger and Collen, 1981). A dose of 10 mg/kg was used since this was the one used to assess fibrinolysis by tPA in rats by almost all published studies (Meden et al., 1993; Overgaard et al., 1992)

### Neuropathological Examination

Rats were killed 24 hours after surgery with an overdose of chloral hydrate and perfused through the left ventricle with saline followed by fixative (paraformaldeyde 4% in 0.1 M phosphate buffer ph 7.4); brains were then removed from the skull, post-fixed for 3 hours in the same solution, then infiltrated overnight in 30% sucrose for cryoprotection, frozen and stored at −20° C. All the collected brains were sectioned on the cryostate in 50 μm-thick serial sections; every sixth slice was mounted on gel-coated slides and stained with cresyl violet for histological evaluation of infarction area.

### Results

### Mortality Rate

Thrombolytic treatment with t-PA caused significant bleeding soon after its infusion, with haemorrhage seriously impairing the clinical outcome. Many animals died within a few hours following the tPA infusion. However, the mortality rate varied significantly among the groups (Table 1). The highest mortality occurred in group 1 (t-PA only treatment), where 57% of rats died following thrombolytic infusion, whereas in group 2. in which a bolus C1-inhibitor was given prior to the t-PA, the incidence of lethal hemorrage was only 25%. No animal died in M5 group or in group 4 (vehicle only) and 5 (C1 inhibitor only). More details for each group are provided below.

Group 1: t-PA treatment (alone) with the highest mortality, a few minutes after infusion, significant bleeding occurred in all animals. Bleeding usually started 10 to 20 minutes after infusion and its intensity and duration were severe. Rats who died following t-PA infusion showed extensive haemorrhagic infiltration of the cortex and white matter, mostly the corpus callosum and the external capsule right above the striatum boundary (data not shown). Macroscopically, blood infiltration was widely visible on the brain surface, both in the ischemic and in the contralateral hemisphere. Oedema and blood infiltration distorted the normal brain architecture, with the contralateral intact hemisphere being compressed by the ischemic one. On the other hand, animals that survived the t-PA treatment there was gross hematoma formation on the surface of the brain (mostly above the ischemic core and in the interhemispheric sulcus) but parenchymal blood infiltration was limited to the region surrounding the pale necrotic ischemic cortex (data not shown).

Group 2: From a clinical perspective, the outcome in t-PA+C1-inhibitor treated rats was significantly better in terms of survival and brain haemorrhage. Bleeding was shorter and significantly less intense and stopped about one hour from its onset. Consequently, there was less impairment of rats' general functional state and mortality was less (only two rats died). Histologically, the two rats with massive bleeding had a severe disruption of cerebral ischemic cortex and underlying structures, due to blood infiltration. Blood was detected in the boundary between external capsule and basal ganglia; ventricular cavities were largely invaded by blood on the ischemic hemisphere, as well as the brain surface. The great majority of treated animals, however, showed milder impairment in brain cytoarchitecture. Commonly, a thin blood epidural infiltration but no hematoma was detected on the brain surface. Although diffuse intraparenchymal blood infiltration was observed within the ischemic area and surrounding structures, no infiltration occurred below it, neither in the white matter nor in the striatum.

Group 3: No animal died following M5 infusion, though some bleeding occurred also in this group. Bleeding onset was observed 15 to 20 minutes after thrombolysis, but was very brief and weaker compared to t-PA treated animals. The general condition of the animals was not impaired by the bleeding and the clinical outcome was good in all infused animals. In one rat, a thick surface hematoma just above ischemic core was observed, but in the remainder no epidural blood infiltration was detected. However, the ventricles were usually not infiltrated. Only one animal showed diffuse ischemic cortex and white matter haemorrhage, with severe contralateral hemisphere compression, nevertheless the clinical outcome was good.

Groups 4 and 5: Both the vehicle group and the “C1 inhibitor only” animals showed typically no bleeding after treatment. The ischemic area was easily detectable after cresyl violet staining as a pale necrotic region surrounded by intact, violet stained tissue; ischemic damage was typically observed within the cortical layer and the internal capsule, with the striatum being usually preserved by ischemic damage. All infarcts were anemic and almost irregularly shaped, but clearly demarcated by a neuropathological borderzone of surviving cell within the ischemic penumbra.

All publications and patents mentioned in the above specification are herein incorporated by reference. Various modifications and variations of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention are intended to be within the scope of the following claims.

### Table 1

Summary table relative to the clinical and histopathological outcome following infusion in the different experimental groups.

| | | | Mortality | |
|---|---|---|---|---|
| Drug | Dose | | rate | Histological appearance |
| Rt-PA | 10 | mg/kg | 57% | Diffuse blood infiltration |
| | | | | within the ischemic cortex, |
| | | | | predominantly on the |
| | | | | surface and in the |
| | | | | parenchyma |
| Rt-PA | 10 | mg/kg | 25% | Diffuse blood infiltration |
| C1-inhibitor | 100 | U/kg | | within the ischemic cortex, |
| | | | | predominantly on the |
| | | | | surface and in the |
| | | | | parenchyma |
| M5 | 15 | mg/kg | 0% | Limited blood infiltration |
| C1-inhibitor | 100 | U/kg | | within the parenchyma; |
| | | | | frequent surface haematome |
| Saline | 1 | ml | 0% | No haemorrhage |
| C1-inhibitor | 100 | u/kg | 0% | No haemorrhage |
| only | | | | |

1. A method of treating, comprising:
a) providing:
i) a subject having symptoms of ischemic stroke;
ii) a first solution comprising C1-inhibitor, and
iii) a second solution comprising tissue plasminogen activator;
b) infusing said subject with at least a portion of said first solution; and
c) infusing said subject, after step b), with at least a portion of said second solution, under conditions wherein said symptoms are reduced.

2. The method of claim 1, wherein said infusing comprises intravenous infusion.

3. The method of claim 1, wherein said infusing comprises intrarterial infusion.

4. The method of claim 1, wherein said tissue plasminogen activator is selected from the group consisting of native t-PA and mutated t-PA.

5. The method of claim 1, wherein said C1-inhibitor is recombinantly produced.

6. A method of reducing the risk of intracranial bleeding from therapeutic thrombolysis, comprising:
a) providing:
i) a subject having symptoms of ischemic stroke;
ii) a first solution comprising tissue plasminogen activator, and
iii) a second solution comprising C1-inhibitor;
b) infusing said subject with at least a portion of said second solution prior to said first solution, in order to reduce the risk of intracranial bleeding caused by tissue plasminogen activator; and
c) infusing said subject, after step b), with at least a portion of said first solution.

7. The method of claim 6, wherein said infusing comprises intravenous infusion.

8. The method of claim 6, wherein said infusing comprises intrarterial infusion.

9. The method of claim 6, wherein said tissue plasminogen activator is selected from the group consisting of native t-PA and mutated t-PA.

10. The method of claim 6, wherein said C1-inhibitor is recombinantly produced.

11. A formulation, comprising a mixture of tissue plasminogen activator and C1-inhibitor, said mixture formulated for infusion.

12. The formulation of claim 11, wherein said tissue plasminogen activator is selected from the group consisting of native t-PA and mutated t-PA.

13. The formulation of claim 11, wherein said C1-inhibitor is recombinantly produced.
